(19) United States
(12) Patent Application Publication
(10) Pub. No.: US 2024/0299260 A1
(43) Pub. Date: Sep. 12, 2024

(54) COMPACT CARE AND/OR MAKEUP POWDER

(71) Applicant: L V M H RECHERCHE, Saint Jean De Braye (FR)

(72) Inventors: Murielle Farigoule, Saint Jean De Braye (FR); Valérie Bouchard De La Poterie, Saint Jean De Braye (FR)

(21) Appl. No.: 18/027,156

(22) PCT Filed: Sep. 21, 2021
(86) PCT No.: PCT/FR2021/051614

§ 371 Date: Mar. 20, 2023

(30) Foreign Application Priority Data
Sep. 21, 2020 (FR) 2009561

(51) Int. Cl. A61K 8/19; A61K 8/02; A61K 8/29; A61K 8/55; A61Q 1/10; A61Q 1/12

(52) U.S. Cl. CPC A61K 8/19; A61K 8/022; A61K 8/29; A61K 8/55; A61Q 1/10; A61Q 1/12; A61K 2800/43; A61K 2800/612

(57) ABSTRACT

The present invention relates to a solid cosmetic composition for caring for and/or making up keratin materials, in particular the skin, in the form of a compact powder comprising, in a physiologically acceptable medium: a solid phase comprising at least fillers and pigments treated with an additive of formula (I) [Chem 1], it being possible to obtain said composition by means of a process comprising the following steps: (i) mixing a liquid phase comprising at least water and the solid phase in powder form, so as to form a paste; and (ii) shaping the paste by compacting and total or partial removal of the liquid phase so as to obtain the composition in the form of a compact powder.

### FIELD OF THE INVENTION

The present invention relates to the cosmetics field and in particular to the field of compact powders, particularly intended for cosmetic care and/or for making up the skin.

### STATE OF THE ART

Compact powders for make-up and/or skin care can be prepared by the “dry” process or by the “wet” process if a solvent or a diluent is used to disperse the powders. Using a dry process, pulverulent materials such as fillers and pigments are mixed with a binder essentially composed of non-volatile fatty substances. By wet process, the pulverulent materials and the binder are diluted in a solvent to obtain a paste (otherwise called slurry) which is then dried. By dry process, the compact powders are generally prepared by mixing all the components of the pulverulent phase then by adding to this mixture a binding fatty phase with stirring. The mixture is then crushed, sieved, then poured into a dish and compacted. The compaction is typically carried out at a pressure of 5 to 25 MPa. By wet process, the manufacturing process uses a paste (slurry) and does not comprise a dry compaction step. The trend of creamy powders, with improved sensory and comfort properties compared to conventional powders, is highly sought and that is why slurry is an interesting and up-to-date technology. ‘Slurry’ is a term used to designate a cosmetic paste (suspension) composed of a particulate phase (fillers and pigments), a non-volatile liquid phase (binder to provide smoothness to the powder and a creamy side) and a solvent, which will be evaporated during the process.

The most commonly used solvent is isododecane but due to technological and environmental developments, water is now preferred because it is a solvent that brings fewer technological risks.

However, the use of water in a slurry process aiming at manufacturing make-up products with pigments can generate formulation incompatibilities or product surface inhomogeneities, such as marbling or other color irregularities, which give a visual aspect less perfect and less attractive for consumers.

The present invention therefore aims at providing new solid cosmetic make-up compositions, in particular compact make-up powders, which have a homogeneous appearance, in particular when they are produced by a water slurry process.

“Homogeneous” means a composition in which the various constituents and in particular the pigments are distributed to give a uniform or substantially uniform appearance to the naked eye. In particular, the composition obtained according to the process of the invention does not have surface color irregularities, such as marbling.

### DISCLOSURE OF THE INVENTION

A first aspect of the invention is therefore a solid cosmetic composition for caring for and/or making up keratin materials, in particular the skin, in the form of a compact powder comprising, in a physiologically acceptable medium:
a solid phase comprising at least:
fillers and
pigments treated with an additive of formula (I)

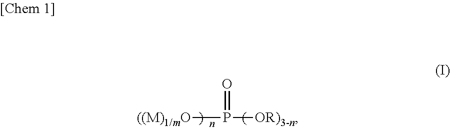

in which:
   n represents 1 or 2,
   M represents H or a cation,
   m represents 1 when M is H and m represents the valence of the cation when M is a cation,
   R represents:
   a group G selected from a saccharide or a group-[CH₂—CHR₁—O]_{q}—R₂ or —[CH₂—CH(CH₂OH)—O]_{q}—R₂ where:
   q represents an integer from 1 to 1000,
   for each CH₂—CHR₁—O unit, R1 independently represents H or a methyl,
   R₂ represents H or an alkyl comprising from 1 to 3 carbon atoms, and,
   a hydrocarbon chain comprising from 1 to 500 carbon atoms substituted by one or more G, phosphate (of formula OPO3(M)_{2/m}) and/or hydroxyl (OH) groups,
said composition being capable of being obtained by a process comprising the following steps: (i) mixing a liquid phase comprising at least water and the solid phase in powder form, so as to form a paste; and (ii) shaping the paste by compacting and total or partial removal of the liquid phase so as to obtain the composition in the form of a compact powder.

It also relates to a cosmetic process for caring for and/or making up keratin materials, in particular the skin, and more particularly the face, comprising the application to the keratin materials of a composition as defined according to the invention.

“Composition according to the invention” generally means the final cosmetic composition obtained according to the process of the invention, to be distinguished from the paste or slurry, which is obtained in an intermediate step of the process.

### DETAILED DESCRIPTION OF THE INVENTION

A first aspect of the invention is therefore a solid cosmetic composition for caring for and/or making up keratin materials, in particular the skin, in the form of a compact powder comprising, in a physiologically acceptable medium:
a solid phase comprising at least:
fillers and
pigments treated with an additive of formula (I)

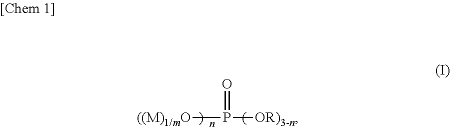

in which:
   n represents 1 or 2,
   M represents H or a cation,
   m represents 1 when M is H and m represents the valence of the cation when M is a cation,
   R represents:
   a group G selected from a saccharide or a group-[CH₂—CHR₁—O]_{q}—R₂ or —[CH₂—CH(CH₂OH)—O]_{q}—R₂ where:
   q represents an integer from 1 to 1000,
   for each CH₂—CHR₁—O unit, R1 independently represents H or a methyl,
   R₂ represents H or an alkyl comprising from 1 to 3 carbon atoms, and,
   a hydrocarbon chain comprising from 1 to 500 carbon atoms substituted by one or more G, phosphate (of formula OPO3(M)_{2/m}) and/or hydroxyl (OH) groups,
said composition being capable of being obtained by a process comprising the following steps: (i) mixing a liquid phase comprising at least water and the solid phase in powder form, so as to form a paste; and (ii) shaping the paste by compacting and total or partial removal of the liquid phase so as to obtain the composition in the form of a compact powder.

“Compact powder” means a solid composition based on powders, distinct from a loose powder, and more particularly in the present case,—a composition comprising at least one solid phase shaped by a step called compaction (compacting) step during which application is made to a paste formed from said solid phase and from a liquid phase comprising at least water, of a pressure intended to provide said solid phase with cohesion such that the composition obtained is solid, namely that it does not flow under its own weight and retains its shape over time. Typically, the pressure applied to the particulate phase.

As opposed to a compact powder, a loose powder is generally dry prepared by simply mixing the ingredients, optionally accompanied by grinding and sieving, without a compaction step aiming at shaping the particulate phase.

Compact powders are generally prepared by mixing all the components of the particulate phase then adding a liquid phase to this mixture with stirring. The mixture is then poured into a cup, bucket or mold and compacted. The compaction is typically carried out at a pressure of 5 to 25 MPa, and results in a reduction in the volume of the compacted composition

A compact powder is different from a “cast” composition which, conversely, consists mainly of waxes and binders. A cast composition is prepared by heating a fatty phase which is solid at room temperature and which needs to be melted in order to be mixed with pulverulent materials.

The composition of the invention will generally have a hardness ranging from 100 to 500 Gf (gram force), in particular from 200 to 400 Gf (gram force). The hardness of a composition can be measured according to the following protocol.

The hardness is measured using a TA.XT PLUS TEXTURE ANALYSER (STABLE MICROSYSTEMS) texturometer, equipped with a rod on which is fixed a cylinder with a diameter of 5 mm which is applied to the surface of the compact. The cylinder penetration force is measured over a thickness of 1 mm at a speed of 0.5 mm/second.

According to a particular embodiment, the composition according to the invention is obtained by a process comprising the following steps: (i) mixing a liquid phase comprising at least water and the solid phase in powder form, so as to form a paste; and (ii) shaping the paste by compacting and total or partial removal of the liquid phase so as to obtain the composition in the form of a compact powder.

‘Partial or total removal of the liquid phase (present in the paste)’ according to the invention, means that the compact powder obtained can be anhydrous (total removal) or comprise a final water rate depending on the desired moisturizing properties. The removed liquid phase comprises water and any additional volatile or non-volatile solvents and binders (oils). The liquid phase can consist of volatile or non-volatile liquids at room temperature.

This removal step is controlled so as to obtain the desired final percentage of water in the composition. Reference will thus be made to ‘controlled removal’ or ‘controlled suction’ in the illustrative examples of the invention.

This step of totally or partially removing the liquid phase allows to substantially remove the liquid phase in order to solidify the composition. The total or partial removal of the liquid phase can be carried out with mechanical means such as suction, or thermal means such as heat.

The removal of the liquid phase can also comprise the combination of the means described previously. Thus, in accordance with a variant of the process of the invention, the removal of the liquid phase contained in the paste comprises a mechanical removal followed by a thermal removal.

The process may for example comprise two successive steps: the partial drying of the paste using mechanical removal means to obtain a wet powder, and the drying of the wet powder by thermal means. The mechanical removal can be carried out by suction under a press, and the thermal removal can be carried out for example by drying in an oven, preferably at a temperature below 70° C., more preferably still below 60° C. According to a particular embodiment, the total or partial removal of the liquid phase is done by suction under vacuum, at room temperature and in particular with a compression pressure between 1 and 4 bar and a suction pressure between 1 and 4 bar (limits included).

The person skilled in the art will be able to adapt the duration of this step to optimize the total or partial removal of the liquid phase. According to a particular embodiment, the total or partial removal of the liquid phase is done by suction under vacuum, at ambient temperature at a compression pressure of 1 to 2 bar and a suction pressure of 1 to 2 bar, for 3 to 20 seconds, in particular a compression pressure of 2 bar and a suction pressure of 1 bar for 20 seconds.

The paste can comprise a higher or lower proportion of liquid depending on the duration and the means of removal used.

The water thus preferably represents from 10% to 70% by weight of the paste, and from 0% to 35% by weight of the final solid composition according to the invention.

Depending on whether the water is totally or partially removed from the paste to form the solid composition, the water content of the final compact powder can vary from 0 to 50% by weight of the composition.

Thus, the water content of the solid compositions of the invention will generally range from 0% to 50% by weight of water, in particular from 10% to 45% by weight of water relative to the total weight of said composition.

According to a particular embodiment, the composition of the invention is anhydrous, that is to say comprising less than 1% by weight of water and advantageously is free of water (0% water). This is the case where the removal of water from the paste is total. In the case of total removal of the liquid phase, this removal step may comprise a heating step, for example by drying in an oven, preferably at a temperature below 70° C., even more preferably below 60° C.

According to another particular embodiment, the composition of the invention comprises a water content ranging from 1% to 40%, in particular from 5% to 30%, or even from 10 to 20% by weight relative to the total weight of the composition.

### Solid Phase

The solid phase is initially, before the shaping step, in powder form. This is why it will also be designated by the term particulate solid phase or else by the generic term “powder” or else “pulverulent phase” when the solid phase is designated before the shaping step, when said phase solid is mixed with a liquid phase to form a paste.

This solid phase consists of a mixture of particles of different shapes, not being limited to the following shapes: rods, platelets and sphericals. According to a particular embodiment, the fillers in the form of platelets are in high proportion in the cosmetic formulas of the invention. To these white fillers can be added texture and touch agents such as spherical fillers to provide glide. The aim is to optimize the ratio of the different powders so that they compact while maintaining satisfactory sensoriality (glide, pick-up and softness properties) and the desired shade.

The particulate solid phase advantageously consists of a mixture of pulverulent materials comprising at least one pigment and/or at least one filler. The pigments can be selected from mineral pigments, organic pigments and pearlescent pigments.

Pigments Treated with an Additive

The mineral pigments can be selected from iron oxides, in particular black, yellow, red and brown iron oxides; titanium dioxide, manganese violet; ultramarine blue; chromium oxides, in particular hydrated chromium oxide, ferric blue, carbon black, and mixtures thereof.

Among the organic pigments, mention may in particular be made of the lakes obtained from dyes such as the dyes D&C Black No. 2, FD&C Blue No. 1, FD&C Green No. 3, D&C Green No. 5, D&C Orange No. 4, D&C Orange No. 5, D&C orange No. 10, D&C No. red 3, D&C Red No. 6, D&C Red No. 7, D&C red No. 9, D&C red No. 13, D&C red No. 19, D&C Red No. 21, D&C Red No. 22, D&C Red No. 27, D&C Red No. 28, D&C Red No. 30, D&C Red No. 33, D&C Red No. 36, FD&C Red No. 40, FD&C Yellow No. 5, FD&C Yellow No. 6, D&C Yellow No. 10 and Cochineal Carmine.

The pearlescent pigments are for example selected from mica covered with titanium oxide, mica-titanium covered with iron oxide, mica-titanium covered with ferric blue, mica-titanium covered with chromium oxide, or tin oxide; mica-titanium covered with an organic pigment as described previously, as well as pigments based on bismuth oxychloride.

The pigments used according to the invention are pigments treated with a particular additive as described below.

Thus, the composition of the invention will comprise pigments or a pigmentary composition comprising a pigment and an additive of formula (I)

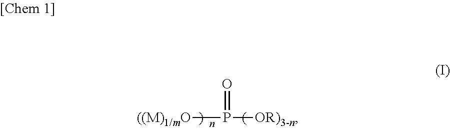

in which:
   n represents 1 or 2,
   M represents H or a cation,
   m represents 1 when M is H and m represents the valence of the cation when M is a cation,
   R represents:
   a group G selected from a saccharide or a group-[CH₂—CHR₁—O]_{q}—R₂ or —[CH₂—CH(CH₂OH)—O]_{q}—R₂ where:
   q represents an integer from 1 to 1000,
   for each CH₂—CHR₁—O unit, R1 independently represents H or a methyl,
   R₂ represents H or an alkyl comprising from 1 to 3 carbon atoms, and,
   a hydrocarbon chain comprising from 1 to 500 carbon atoms substituted by one or more G, phosphate (of formula OPO3(M)_{2/m}) and/or hydroxyl (OH) groups.

Such pigments are described in particular in the international application WO2012120098 from SENSIENT.

This pigmentary composition is suitable for introduction into a cosmetic composition, in particular aqueous cosmetic composition. It is generally in the form of a powder comprising the pigment and the additive intimately mixed, the additive generally being adsorbed or precipitated on the surface of the pigment. A pigmentary composition in powder form is particularly advantageous compared to a composition in liquid form (suspension, emulsion or solution).

It turns out that the pigment compositions described in application WO2012120098 disperse very easily and effectively in an aqueous medium or in the continuous aqueous phase of an oil-in-water emulsion.

In this compound of formula (I), it is meant that the hydrocarbon chain comprises from 1 to 500 carbon atoms, in particular from 1 to 50, typically from 1 to 10 carbon atoms, preferably from 1 to 5 carbon atoms. Hydrocarbon chains can be linear, branched or cyclic. The preferred hydrocarbon chains are alkyl groups (preferably having 1 to 10 carbon atoms, in particular 1 to 5 carbon atoms, preferably 1 to 3, such as methyl, ethyl, n-propyl and isopropyl groups), alkenyl (preferably having 2 to 10 carbon atoms, in particular 2 to 6), aryl (preferably having 6 to 10 carbon atoms), arylalkyl (preferably having 7 to 10 carbon atoms) or alkylaryl (preferably having 7 to 10 carbon atoms). M can in particular be an inorganic cation, such as Ag³⁺, Al³⁺, Fe³⁺, Fe²⁺, Ag²⁺, Zn²⁺, Sn²⁺, Ca²⁺, Ba²⁺, Ag⁺, Na⁺ or an organic cation, such as a diethanolammonium (DEA) (H₃N⁺—(CH₂)₂—OH) or a quaternary ammonium.

According to a particular embodiment, the pigment or pigmentary composition comprises an additive of formula (II) below:

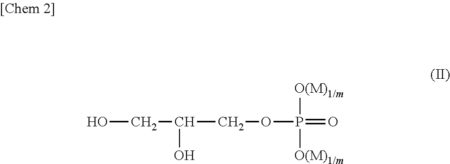

In which M and m are as defined above, (which corresponds to an additive of formula (I) in which n represents 2, R represents a group G of formula-[CH₂—CH(CH₂OH)_{q}—O]q-R₂ where q represents 1 and R2 represents H.

According to a particular and preferred embodiment, a glycerophosphate additive of the following formula (III) is used:

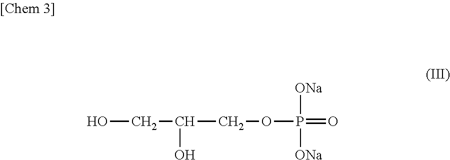

which corresponds to an additive of formula (II) in which M represents Na and m represents 1, this additive being advantageously commercially available.

Thus, according to a particular and preferred embodiment, the pigments used according to the invention are pigments treated with sodium glycerophosphate (otherwise called ‘SGP’ pigments), in particular iron oxides treated with such an additive.

Mention may in particular be made of the pigments treated with a sodium glycerophosphate (SGP) from SENSIENT marketed according to the following references:
Unipure red LC381 SGP (iron oxides, CI77491 and CI 77499 and sodium glycerophosphate)
Unipure black LC989 SGP (iron oxides, CI 77499 and alumina and magnesium oxide and sodium glycerophosphate)
Unipure yellow LC182 SGP (iron oxides, CI 77492 and sodium glycerophosphate)
Unipure white LC 981 SGP (titanium dioxide, CI 77891 and sodium glycerophosphate).

According to another particular embodiment, an additive of formula (V) is used

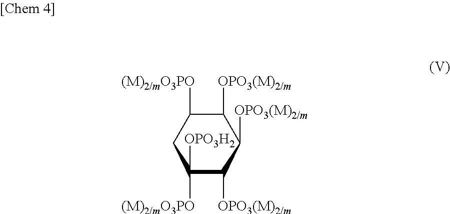

which corresponds to an additive of formula (I) in which n represents 2, R represents a cyclohexyl hydrocarbon chain substituted in positions 2, 3, 4, 5 and 6 by a phosphate group of formula OPO₃H₂. And preferably phytic acid of formula (V′)

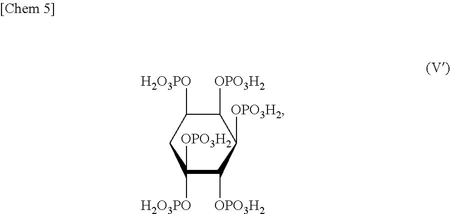

Which corresponds to an additive of formula (V) in which M represents H and m represents 1, this additive being advantageously commercially available, for example from Nutriscience®.

The pigments treated with a phytic acid solution of formula (V′) are described in application WO2012/120098 and FR3080116 from Sensient.

Mention may in particular be made of the pigments treated with a phytic acid (PHY) from SENSIENT marketed according to the following commercial references:
Unipure red LC388 PHY (red iron oxide, CI 77491 and phytic acid and sodium hydroxide)
Unipure black LC 998 PHY (CI 77499 and phytic acid and sodium glycerophosphate)
Unipure yellow LC 188 PHY (CI 77492 and phytic acid and sodium glycerophosphate)
Unipure white LC 985 PHY (anastase, CI 77891 and phytic acid and sodium glycerophosphate).

The composition of the invention will comprise pigments treated with an additive of formula (I), (II) (III), (V) or (V′), preferably a sodium glycerophosphate additive of formula (III), or an additive phytic acid of formula (V′), in a content ranging from 0.5% to 60%, in particular from 0.8% to 50% and preferably from 1% to 30% by weight relative to the total weight of said composition.

According to a particular embodiment, for care products, the total pigment content will generally range from 0.5% to 5% by weight relative to the total weight of the composition.

According to another particular embodiment, for makeup products, the total pigment content will generally range from 8% to 30% by weight, in particular from 12% to 20% by weight relative to the total weight of the composition.

### Fillers

The fillers can be mineral or organic, and of any shape, platelet, spherical or oblong.

According to a particular embodiment, the fillers are selected from platelet fillers, spherical fillers and mixtures thereof.

The fillers advantageously have an average diameter greater than 100 nanometers and less than 200 micrometers, and more particularly a diameter comprised between 5 and 150 micrometers.

According to a variant implementation of the invention, the fillers can be treated so as to modify their surface condition. The fillers used can advantageously be surface-treated to make them hydrophobic or hydrophilic, while guaranteeing sufficient cohesion of the powder of the invention. According to a particular embodiment, the fillers can be treated in the same way as the aforementioned pigments, with a hydrophilic additive as described above.

The fillers are selected in particular from inorganic fillers such as:
talc preferably in the form of particles generally of dimensions less than 40 μm;
micas of natural or synthetic origin, sericites, having dimensions of 2 to 200 micrometers (μm), preferably of 5 to 70 μm and a thickness of 0.1 to 5 μm, preferably of 0.2 to 3 μm;
kaolin having particle sizes generally less than 30 μm;
metallic soaps derived from organic carboxylic acids having from 8 to 22 carbon atoms, preferably from 12 to 18 carbon atoms, for example zinc stearate, magnesium stearate or lithium stearate, zinc laurate, magnesium myristate, preferably in the form of particles having dimensions of less than 10 μm; these metal soaps play in particular the role of cohesive agent in the powder;
zinc oxides; calcium carbonate; barium sulphate; magnesium carbonate, magnesium hydrocarbon, silica, glass beads, ceramic beads; boron nitride;
and mixtures thereof.

As organic fillers, mention may in particular be made of:
crosslinked or non-crosslinked starches, for example corn, wheat, rice or potato starches;
cellulose powders;
synthetic polymer powders, crosslinked or not, spheronized or not, expanded or not, such as polyamide powders (for example poly-R-alanine powders and nylon powders such as those marketed under the name ORGASOL®), poly(meth)acrylic acid or poly(meth)acylate powders such as cross-linked methyl methacrylate powders, silicone resin powders such as silsesquioxanes,
and mixtures thereof.

According to a particular embodiment, the composition of the invention will comprise at least fillers playing the role of excipient, fillers playing a role on the texture and the feel and fillers playing a cohesion role within the composition.

Thus the composition of the invention will advantageously comprise:
A filler selected from the group consisting of talc, mica, sericite, synthetic fluorophlogopite, boron nitride, barium sulfate, amino acids, starches, and mixtures thereof, as an excipient agent;
A filler selected from the group consisting of silicas, silicone resin, PMMA, cellulose, and Nylon-12, as texture or feel agent, and
A filler selected from the group of metallic soaps derived from organic carboxylic acids having from 8 to 22 carbon atoms, preferably from 12 to 18 carbon atoms, preferably selected from zinc stearate, magnesium stearate, magnesium myristate and mixtures thereof.

Fillers can also be classified according to their shape. The composition will advantageously comprise spherical fillers and platelet fillers.

According to a particular embodiment, the platelet fillers are selected from the group consisting of talc, mica, sericite, synthetic fluorphlogopite, boron nitride, barium sulphate, amino acids and mixtures thereof and the spherical fillers are selected from the group consisting of silica, PMMA particles, cellulose particles, nylon particles, silicone resin particles, starches and mixtures thereof.

Thus the composition of the invention may advantageously comprise at least:
A filler selected from the group consisting of talc, mica, sericite, synthetic fluorophlogopite, boron nitride, barium sulfate, amino acids, and mixtures thereof, as platelet filler; and
A filler selected from the group consisting of silicas, silicone resin, PMMA, cellulose, and Nylon-12, starches and mixtures thereof as a spherical filler.

The content of spherical fillers and platelet fillers can be adjusted according to the desired properties. In particular, the composition may comprise a content ranging from 0.1 to 50%, in particular 1 to 25% by weight, of spherical fillers selected from the group consisting of silica, silicone resin, PMMA, cellulose, Nylon-12, and mixtures thereof, and a content ranging from 0.1% to 70%, in particular 1% to 60%, of platelet fillers.

According to a particular and preferred embodiment, the composition also comprises at least one filler selected from metallic soaps derived from organic carboxylic acids having from 8 to 22 carbon atoms, in a content ranging from 0% to 10% by weight with respect to the total weight of said composition, to improve the cohesion of the powder obtained according to the invention.

### Liquid Phase

The liquid phase comprises at least water.

It may further advantageously comprise a hydroxylated compound selected from polyols, glycol ethers and mixtures thereof.

Depending on whether the water is totally or partially removed from the paste to form the solid composition, the water content of the final compact powder can vary from 0 to 50% by weight of the composition.

Thus, the water content of the solid compositions of the invention will generally range from 0% to 50% by weight of water, in particular from 10% to 45% by weight of water relative to the total weight of said composition.

According to a particular embodiment, the composition of the invention is anhydrous, that is to say comprising less than 1% by weight of water and advantageously is free of water (0% water). This is the case where the removal of water from the paste is total.

According to another particular embodiment, the composition of the invention comprises a water content ranging from 1% to 40%, in particular from 5% to 30%, or even from 10 to 20% by weight relative to the total weight of the composition.

The presence of water in the compact powder allows to obtain, at the time of application of the composition to the skin, a feeling of freshness resulting from the almost instantaneous evaporation of the water upon contact with the skin. This surprising effect is particularly sought in the case of compositions applied to the face because it provides a feeling of well-being sought by the consumer who uses this type of product.

The liquid phase can advantageously also comprise at least one hydroxylated compound selected from a polyol, a polyol ether and mixtures thereof.

Among the polyols and glycol ethers, mention may in particular be made of:
C3-C6 polyols, for example a C3-C6 aliphatic diol (or glycol) such as propane-1,2-diol (or propylene glycol), propane-1,3-diol, a butylene glycol, pentylene glycol (pentane-1,5-diol), or a triol such as glycerol,
a glycol ether or an aromatic alcohol such as phenoxyethanol,
   and mixtures thereof.

Preferably, the composition comprises at least one C3-C5 polyol, preferably glycerol.

The total content of polyol(s) and/or glycol ether(s) in the composition of the invention will in particular range from 1% to 30% by weight relative to the total weight of the composition, in particular from 1% to 25% by weight relative to the total weight of the composition. According to a particular embodiment, the total content of polyol (s) and/or glycol ether(s), in particular of glycerol, ranges from 5% to 25% by weight, preferably from 5% to 15% by weight relative to the total weight of the composition.

### Oily Phase (Optional)

The composition may also optionally comprise an oily phase comprising at least one oil.

“Oily phase” means an oil or a mixture of oils which are miscible with each other.

“Oil” means, within the meaning of the invention, a fatty substance, insoluble in water, liquid at 25° C. and atmospheric pressure.

These oils can be volatile or non-volatile, and of vegetable, mineral or synthetic origin.

The oily phase can comprise binders of various kinds such as fatty esters, silicones, hydrocarbon oils, butters, and mixtures thereof. According to a particular embodiment, use will be made of fatty esters, hydrocarbon oils, and mixtures thereof.

These ingredients play the role of binders of the particulate solid phase at the time of compaction of the paste and provide cohesion, and in terms of texture, creaminess, comfort when applied and a care side, non-drying to the powder.

As hydrocarbon oils, mention may in particular be made of linear or branched C₈-C₁₆ alkanes, branched C₈-C₁₆ esters, hydrocarbon-based oils of plant origin, C₁₀-C₄₀ synthetic ethers, C₁₀-C₄₀ synthetic esters, C₁₂-C₂₆ fatty alcohols, C₁₂-C₂₂ higher fatty acids, and mixtures thereof.

Mention may in particular be made of isononyl isonanoate, pentaerythrityl triisostearate, isohexyl neopentanoate or a hydrocarbon such as isododecane, isodecane, isohexadecane, n-dodecane (C₁₂) and n-tetradecane (C₁₄) or the undecane-tridecane mixture.

As silicone oils, mention may in particular be made of linear or cyclic silicone oils, phenyl or non-phenyl silicone oils, and mixtures thereof.

### Structuring Agents

The composition of the invention may optionally comprise agents for structuring or gelling the liquid phase, in particular hydrophilic gelling agents (or suspending agents), pasty fatty substances or waxes, of animal, vegetable, mineral or synthetic origin.

By way of “hydrophilic gelling agents”, mention may in particular be made of acrylic acid polymers, polysaccharide gelling agents such as alginates, xanthan gums, carrageenan gums, agar gums, guar gums, gellan gums, sclerotium gums, chitosans, mannans, cellulose derivatives, gelatin, pectins, mineral gelling agents such as bentones or modified silicas, and mixtures thereof.

According to a particular embodiment, the solid composition also comprises at least one hydrophilic gelling agent selected from a bentonite gel, a hectorite gel, and mixtures thereof.

### Film Forming Agents

The composition can advantageously comprise a film-forming agent, preferably a film-forming polymer.

Film-forming polymer means a polymer capable of forming a continuous film on a support. In the text, the word polymer can denote a homopolymer or a copolymer. “Copolymer” means a polymer comprising at least two monomers or two different blocks, which may be of the same chemical family but of different structure.

The film-forming agents have an ability to form a film on the surface of the skin at the time of application of the composition, and allow to obtain an improvement in the staying power over time and in the resistance to sebum, to perspiration or mechanical stresses, in particular related to facial movements. The formation of this film thus allows to improve and extend the staying power of the composition, and therefore the effect produced by said composition, but also to give it a property called “non-transfer” property.

The compositions according to the invention comprising at least one such film-forming agent are referred to as “long-lasting” and/or “non-transferring”.

These film-forming agents can be dissolved in a liquid phase according to their affinity or else be dispersed in the form of particles or latex in a phase in which they are insoluble. These film-forming agents are advantageously polymers which can be hydrophilic in nature and thus have an affinity for the aqueous phase, or hydrophobic in nature and have an affinity for the optional oily phase of the composition.

The hydrophilic character is in particular conferred by the presence of oxygen or nitrogen atoms in the molecule, in the form of hydroxylated, carboxylic, acrylic, ester, amide, urethane or other groups comprising heteroatoms or else an ether or ketone function.

The hydrophobic character is related to the absence of oxygen or nitrogen atoms and more generally of heteroatoms in the molecule, and the exclusive presence of saturated or unsaturated, cyclic, linear or branched hydrocarbon groups.

Hydrophilic film-forming agents can advantageously be selected from synthetic compounds such as polyurethanes and their derivatives, and more particularly aqueous polyurethane dispersions which may have a solid content (dry matter) of 20 to 60% by weight, in particular polyurethane-35, polyalkylenes, such as polyisoprene.

Such polyurethane dispersions are for example Polyurethane-34 sold under the commercial references Baycusan® C1000, C1001 by the company BAYER, and Polyurethane-35, which comprises 41% of polyurethane in dry matter in dispersion in water, sold under the commercial reference Baycusan® C1004 by the company BAYER (COVESTRO).

It is also advantageously possible to choose hydrophilic film-forming agents from compounds that are natural or of natural origin, such as native or chemically modified polysaccharides, and in particular pullulan or one of its derivatives, a starch or one of its derivatives and mixtures thereof.

According to a particular embodiment, the composition comprises a hydrophilic film-forming polymer, advantageously selected from native or chemically modified polysaccharides.

According to a particularly preferred variant, the film-forming polymer is selected from pullulan or its derivatives, a starch or its derivatives, in particular hydroxypropyl starch. A particularly preferred starch derivative is hydroxypropyl starch (INCI Hydroxypropyl Starch).

According to a particularly preferred variant, the composition comprises at least one polyol or one polyol ether and at least one hydrophilic film-forming agent.

The hydrophilic film-forming polymer may be present in a content ranging from 0.1 to 10% by weight, preferably from 0.5 to 5% by weight of dry matter relative to the total weight of said composition. The percentage of hydrophobic film-forming polymer is expressed as % by weight of dry extract (dry matter or active matter, a.m) relative to the total weight of the composition.

The composition may comprise at least one hydrophobic polymer, to improve the staying power properties of the film of the composition on the skin.

Hydrophobic or fat-soluble film-forming polymer means a film-forming polymer dissolved in the optional oily phase of the composition.

The hydrophobic film-forming polymer can be of natural or synthetic origin, and is advantageously selected from the group consisting of:
trimethylsiloxysilicates such as Belsil TMS 803 (WACKER), KF7312 from Shin Etsu
phenylalkylsiloxysilicates in which the alkyl group preferably comprises from 1 to 6 carbon atoms, such as phenylpropyldimethylsiloxysilicate,
silicone acrylate polymers such as acrylate/dimethicone copolymers, and in particular acrylate/dimethicone copolymers in cyclopentasiloxane (such as, for example, KP-545 from Shin-Etsu), acrylate/dimethicone copolymers in methyl trimethicone (such as, for example, KP-549 and KP-579 from Shin-Etsu), and acrylate/dimethicone copolymers in isododecane (such as, for example, KP-550 from Shin-Etsu); acrylate/polytrimethylsiloxy-methacrylate copolymers, and in particular acrylate/polytrimethylsiloxy-methacrylate copolymers in dimethicone (such as, for example, FA-4003 DM from Dow Corning®), acrylate/polytrimethylsiloxy-methacrylate copolymers in isododecane (such as for example the FA-4004 ID from Dow Corning®),
polyalkylsilsesquioxanes comprising from 1 to 6 carbon atoms, and preferably polymethylsilsesquioxane (such as for example Silform® Flexible Resin from Momentive),
trialkylsiloxysilylcarbamoyl pullulans in which the alkyl group comprises 1 to 6 carbon atoms, and preferably trimethylsiloxysilylcarbamoyl pullulan (such as TSPL-30-ID from Shin-Etsu),
copolymers of vinylpyrrolidone (VP) and alkene comprising from 2 to 20 carbon atoms, such as copolymers of VP/eicosene, VP/hexadecene, VP/styrene,
copolymers of a vinyl ester, and preferably copolymers of vinyl acetate/allyl stearate, vinyl acetate/vinyl laurate, vinyl acetate/vinyl stearate, vinyl acetate/octadecene, vinyl acetate/octadecylvinylether, vinyl propionate/allyl laurate, vinyl propionate/vinyl laurate, vinyl stearate/octadecene-1, vinyl acetate/dodecene-1, vinyl stearate/ethyl vinyl ether, vinyl propionate/cetyl vinyl ether, vinyl stearate/allyl acetate, dimethyl-2,2-vinyl octanoate/vinyl laurate, dimethyl-2,2-allyl pentanoate/vinyl laurate, dimethyl vinyl propionate/vinyl stearate, allyl dimethyl propionate/vinyl stearate,
hydrogenated or non-hydrogenated polyolefins, and preferably polymers or copolymers of alkenes comprising from 2 to 20 carbon atoms, such as polybutenes, polyisobutenes, polydecenes,
alkylcelluloses, and preferably alkylcelluloses carrying an alkyl group comprising from 2 to 6 carbon atoms, such as ethylcellulose and propylcellulose,
polyvinyl alcohols,
   and mixtures thereof.

The hydrophobic film-forming polymer may be present in a content ranging from 0.1 to 10% by weight, preferably from 0.5 to 5% by weight of dry matter relative to the total weight of said composition. The percentage of hydrophobic film-forming polymer is expressed as % by weight of dry extract (dry matter or active matter, a.m) relative to the total weight of the composition.

### Additional Ingredients

The composition may further comprise at least one additional ingredient selected from dyes, surfactants, perfumes, electrolytes, antioxidants, preservatives, and physical and/or chemical UV filters.

The composition of the invention can advantageously incorporate any type of cosmetic active agent, whether hydrophilic or lipophilic. The preferred active agents are selected from the group consisting of active agents having a moisturizing activity such as hyaluronic acid or ceramides, active agents having an anti-aging activity; active agents having a smoothing activity and on the skin radiance the active agents having a skin depigmenting activity or lightening activity; active agents having a slimming activity; active agents having a moisturizing activity; active agents having a soothing or relaxing activity; and mixtures thereof.

The composition is advantageously devoid of surfactant, because it is not necessary, unlike the powders of the prior art obtained by pasting powdery materials with water, to use a surfactant to guarantee a good homogeneity of the paste. Thus, according to a particular embodiment, the surfactant content in the composition of the invention will be less than 5% by weight, in particular less than 3% by weight, preferably less than 1% by weight relative to the total weight of the composition.

According to a particular embodiment, the composition of the invention does not comprise a surfactant.

### Wet Preparation Process (Formation of an Intermediate Paste ‘Slurry’)

The composition of the invention based on powders is produced by the wet process by preparing a paste (or pasty composition) according to a suitable industrial process.

According to one of its aspects, the object of the invention is a process for manufacturing the powder-based composition described above, which process comprises: (i) mixing a liquid phase comprising at least water and the solid phase in powder form comprising fillers and treated pigments as defined in the invention, so as to form a paste; and (ii) shaping the paste, in particular in a silicone mold, by compacting and total or partial removal of the liquid phase present in the paste.

Thanks to the presence of water for the embodiments with partial removal of the liquid phase present in the paste, it is possible to incorporate in the composition of the suspending agents which will allow to preserve the paste for a certain time in storage phase so that the pigments remain in suspension, that there is no release and that the paste remains homogeneous.

As suspending agents, and as described above, use can advantageously be made of magnesium aluminum silicate, hectorite, bentonite, xanthan gum, chondrus crispus and mixtures thereof.

Thus, a composition of the invention will also comprise at least one suspending agent, in particular selected from magnesium aluminum silicate, hectorite, bentonite, xanthan gum, chondrus crispus and mixtures thereof.

The process of the present invention, using water and pigments treated with an additive of formula (I), (II), (III), (V) or (V′) advantageously allows to obtain a homogeneous paste and homogeneous compact powders. “Homogeneous” means a composition in which the various constituents and in particular the pigments are distributed to give a uniform or substantially uniform appearance to the naked eye. In particular, the composition obtained according to the process of the invention does not have surface color irregularities, such as marbling.

According to another of its aspects, the object of the invention is a process for manufacturing a composition based on powders, which process comprises: (i) mixing a liquid phase comprising at least water and the solid phase in powder form comprising fillers and treated pigments as defined in the invention, so as to form a paste; and (ii) shaping the paste, in particular in a silicone mold, by compacting and total or partial removal of the liquid phase present in the paste, in particular by vacuum suction, optionally followed by freezing, and optionally drying at a temperature below 70° C.

It is implicit that at the time of the removal of the water, part of the oily (volatile or non-volatile) phase can also be removed.

‘Total or partial removal’ according to the invention, means that the compact powder obtained may comprise a residual water rate in the final product. The total or partial removal is carried out, according to a particular embodiment, by suction under vacuum at ambient temperature and with a suction pressure between 1 and 4 bar, a compression pressure between 1 and 4 bar.

The paste (‘slurry’) comprises the various constituents, preferably in the following proportions:
from 15% to 80% by weight of particulate solid phase relative to the total weight of the paste;
10 to 70% by weight of water relative to the total weight of the paste; and
0.1 to 30% by weight of oils and/or structuring agents relative to the total weight of the paste.

The water preferably represents from 10% to 70% by weight, preferably from 15 to 60% by weight of the paste.

For shaping, the paste can be placed in molds in order to undergo shaping according to a technique known to the person skilled in the art. The molds can be made of metal or silicone and can be reused once the composition has been removed from the mold. The molds can also be cups or buckets which remain attached to the composition once manufactured in order to then be placed in the housing of a packaging. When the paste is liquid, it can be injected either through the bottom, the side or the top of the mold or be poured by simply filling the mold through its upper opening. A process for injection through the bottom of the molds is for example known under the name “Back Injection Machine” or BIM. A process for injection from the side is for example known by the name “Side Injection”. A process for filling molds that involves pouring the paste into the mold from above is known as “Top Fill”. The paste can also be more viscous so that it can be dosed by preparing pieces of paste. The percentage of volatile or non-volatile liquid (water and optional solvents and additional volatile or non-volatile binders) is preferably selected such that the paste has a sufficient consistency to be able to be kneaded, measured and cut. In this case, it is necessary to shape the paste so that the composition hugs the edges of the buckets, before removing the volatile liquid. This shaping can be carried out by pressing at the same time as the removal of the volatile or non-volatile liquid. The preparation process of the invention comprises a step of totally or partially removing the volatile or non-volatile liquid, the purpose of which is to substantially remove the volatile or non-volatile liquid in order to solidify the composition. The total or partial removal of the volatile or non-volatile liquid can be carried out with mechanical means such as suction, or thermal means such as heat. According to a particular and preferred embodiment, the total or partial removal of the volatile or non-volatile liquid is done by suction under vacuum, at room temperature and with a suction pressure preferably between 1 and 4 bar, a compression pressure preferably between 1 and 4 bar (limits included).

The person skilled in the art will know how to adapt the duration of this step to optimize the total or partial removal of the volatile or non-volatile liquid. The paste may comprise a higher or lower proportion of volatile or non-volatile liquid depending on the duration and the means of removal used.

The water thus preferably represents from 10% to 70% by weight of the paste, and from 0% to 35% by weight of the final solid composition according to the invention.

The pressing (compacting) is carried out in particular by means of a stamp which contacts the paste and which compresses the composition between the bottom of the mold and its surface during suction. The end of the stamp or the bottom of the mold is perforated to allow the extraction of volatile or non-volatile liquid out of the paste during suction. In this embodiment, a porous material can be deposited on the free surface of the paste in each mold, or be placed on the stamp which serves as a press in order to retain the powder in the mold during suction. This porous material is for example a fabric, a weft or an absorbent sheet. It is removed from the composition once the suction is complete. The removal of the volatile or non-volatile liquid can also comprise the combination of the means described above. Thus, in accordance with a variant of the process of the invention, the removal of the volatile or non-volatile liquid contained in the paste comprises a mechanical removal followed by thermal removal.

The process may for example comprise two successive steps: the partial drying of the paste using mechanical removal means to obtain a wet powder, and the drying of the wet powder by thermal means. The mechanical removal can be carried out by suction under a press, and the thermal removal can be carried out for example by drying in an oven, preferably at a temperature below 70° C., more preferably still below 60° C.

According to a first preferred embodiment, the compositions of the invention are prepared by a process which comprises:
the preparation of the paste,
introducing the paste into a mold, for example by means of a pump and an injection unit which advantageously comprises nozzles allowing the paste to be injected from below or above the molds,
depositing a porous material on the free surface of the paste and/or depositing a plastic grid on the surface before depositing the porous material,
vacuuming the filled molds and totally or partially removing by suction the volatile or non-volatile liquid present in the paste, preferably and maintaining the paste under a press during the suction, and
returning to atmospheric pressure.

The present invention is particularly suitable for the preparation of cosmetic compositions intended for caring for or making up the body and/or the face. The process of the invention allows to prepare a compact powder of homogeneous appearance, in which the various components and in particular the pigments are well dispersed.

### Process in Buckets

According to a first embodiment, called ‘1^{st} generation’ embodiment (1G process), the paste is compacted from above in a bucket.

During compaction, compression and suction take place simultaneously using tools with a perforated head and a compaction pattern.

The compaction fabric is in contact with the pattern. The rest of the solvent is evaporated in an oven for several hours.

Compression is carried out in two steps:
the first compaction allows to partially suck up the solvent the second compaction gives the final shape with patterns on the surface.

### Silicone Mold Process

According to a second embodiment, called 2^{nd} generation embodiment (2G process), the compact powder obtained no longer needs a bucket and has a three-dimensional pattern, more precise than with the previous embodiment. The visuals and reliefs (3D effect, precision, gestures) are new. The 2G process allows to achieve surface volumes greater than 5 mm, much larger than with conventional compaction, as well as a beautiful surface appearance, good pattern definition and gloss. In addition, the surface appearance of the 2G process is much more precise and brighter than the 1G process due to the absence of intermediate compacting during the creation of the relief (pattern present in the silicone mold).

This 2G process also allows to incorporate a high rate of binder compared to a powder formula produced by dry compaction, and to incorporate a high percentage of water in a compact powder, for example more than 20% by weight of the composition, to increase the feeling of freshness and hydration when applied.

### Preparation of the Paste

The paste is made by pasting the powders with the binder and the solvent. The mixing of the phases is carried out using a Rayneri and a blade of the deflocculating type.

The water, the suspending agents (e.g. bentonite and/or hectorite gels), the preservatives, the glycols and any additional oils are homogenized with stirring then the powder phase (particulate solid phase) is gradually incorporated into the rest of the mixture to thereby form a paste, called a paste or slurry. In the case of adding volatile oils, they are incorporated after the addition of the powder phase, at the end.

### Shaping the Powder According to the 2G Process

For the shaping of the compact powder, use is advantageously made of silicone molds. These molds allow to create new surface reliefs and patterns. These reliefs and patterns are represented in negative on the internal face of the mold.

The paste is incorporated into the silicone molds, a support grid is added on the surface to improve the solidity of the compact.

Then a compacting fabric is added then the compaction is carried out from the top of the mold and the solvent is sucked up using a perforated smooth surface tool.

Compaction is carried out, for example, using a Top Fill, Back Fill, Side Injection machine.

During compression, the fabric is no longer in contact with the pattern, thus the pattern created on the surface is more precise and more in relief compared to the process with a bucket.

After compacting, the paste is placed in the freezer in their silicone molds for between 15 minutes and 2 hours to facilitate demolding and to optimize the definition of the design during demolding.

Then, they are optionally placed in an oven at 45° C. or 50° C. for a more or less long period depending on the content of volatile solvent (water) that it is desired to keep in the end in the final product.

For a content between 0 and 35% by weight of water in the final product, the duration may range from a few minutes to 6 hours depending on the means used (furnace, oven at 45° C. or 50° C. or no drying), to modulate the amount of water in the final product.

Thus, according to a particular embodiment, the compositions of the invention are prepared according to the following process, illustrated in the examples below, according to the following steps:
(i) mixing a liquid phase comprising at least water and the solid phase in powder form, so as to form a paste (‘slurry’):
   a. the ingredients of the aqueous phase A are dissolved in an excess amount of water such that phase A is approximately equivalent in weight to the particulate solid phase B;
   b. the ingredients of phase B are mixed then dispersed in phase A using a Rayneri; for example at 1000 revolutions/minutes for 5 to 10 minutes until a homogeneous mixture is obtained;
   c. the optional binding phase C is added last to the pasty dispersion formed by phases A and B. The slurry is homogenized, for example at 1000 revolutions/minute for 15 minutes;
(ii) shaping the paste by compacting and total or partial removal of the liquid phase so as to obtain the composition in the form of a compact powder, according to the following steps:
   a. the paste is incorporated into molds, for example silicone molds, taking care to remove any air bubbles; advantageously a support grid is added on the surface to improve the solidity of the compact;
   b. a compacting fabric is added then compacting is carried out, for example, from above the mold (Top Fill machine) and the solvent (water) is simultaneously sucked up by creating a vacuum in the enclosure (vacuum suction at ambient temperature) with a compression pressure of 1 to 4 bar and a suction pressure of 1 to 4 bar); the fabric no longer being in contact with the pattern in this step, the pattern created on the surface is more precise and more in relief compared to the 1G process;
   c. the molds are then advantageously placed in the freezer for 30 minutes to 1 hour to allow easy unmolding;
   d. after unmolding, and if necessary, the products are placed in an oven at 50° C. for 5 hours or alternatively at 45° C. overnight until the water content that is desired to be retained in the final product is reached.

### Galenic

According to one particular embodiment, the composition of the invention is in the form of a care powder or a make-up powder for the face or the lips, in particular a blush, an eye shadow, a highlighter, a bronzer, a protective foundation powder, a concealer, a lip powder or an eyebrow powder, preferably a foundation powder, a highlighter, a blush or an eye shadow.

According to a particular embodiment, the composition of the invention may have three-dimensional (3D) relief patterns on the surface, bringing out a particular pattern such as a logo, an alphanumeric character or a string of alphanumeric characters, or a representation styled with an object such as a flower.

This aspect in relief, is visually very attractive, is made possible thanks to the use of silicone molds adapted to the desired shape, the use of which is very suitable for the shaping process used for the solid composition of the compact powder type of the invention.

Powders can be applied with a brush, sponge or finger.

### Cosmetic Process

The invention also relates to a cosmetic process for caring for and/or making up keratin materials, in particular the skin, comprising the application to the keratin materials of a composition according to the invention.

Applied to the skin, in particular of the face, the compact powder, in particular obtained by a ‘slurry’ process as described above, provides the consumer with the comfort and sensoriality sought, with a good make-up result.

The invention will now be illustrated in the following non-limiting examples. Unless otherwise indicated, the % are expressed as % by weight of raw material relative to the total weight of the composition.

### EXAMPLES

In the following examples, the terms “SGP TREATED PIGMENTS” and “SGP TREATED PIGMENTS” respectively designate pigments which have received a hydrophilic surface treatment with sodium glycerophosphate (SGP) or phytic acid (PHY), giving said pigments an affinity for hydrophilic media such as an aqueous phase. Their use allows to surprisingly obtain a slurry of powders whose unctuous and fresh texture is particularly pleasant when applied, while retaining its properties of homogeneity and hardness after shaping the slurry in the form of a compact. These treated pigments are, for example, marketed under the brand UNIPURER by the company SENSIENT COSMETIC TECHNOLOGIES.

### Pigments Tested:

Untreated pigments (control)
Sympholight pigments (comparative)
Sympholight RW (CI 77491 and silica)
Sympholight BW (CI 77499 and silica)
Sympholight YW10 (iron hydroxide and silica)
Sympholight WW (titanium dioxide or CI 77891 and silica)
SGP pigments from SENSIENT (invention)
Unipure red LC381 SGP (iron oxides, CI77491 and CI 77499 and sodium glycerophosphate)
Unipure black LC989 SGP (iron oxides, CI 77499 and alumina and magnesium oxide and sodium glycerophosphate)
Unipure yellow LC182 SGP (iron oxides, CI 77492 and sodium glycerophosphate)
Unipure white LC 981 SGP (titanium dioxide, CI 77891 and sodium glycerophosphate)
PHY pigments from SENSIENT (invention)
Unipure red LC388 PHY (red iron oxide, CI 77491 and phytic acid and sodium hydroxide)
Unipure black LC 998 PHY (CI 77499 and phytic acid and sodium glycerophosphate)
Unipure yellow LC 188 PHY (CI 77492 and phytic acid and sodium glycerophosphate)
Unipure white LC 985 PHY (anastase, CI 77891 and phytic acid and sodium glycerophosphate)
Unipure white LC 987 PHY (rutile, CI 77891 and phytic acid and sodium glycerophosphate)

In accordance with the process of the invention, in the following examples; the aqueous phase A comprises an excess of water, at least part of which is removed at the time of the solvent suction step.

The initial amount of water must therefore be calculated to obtain the intermediate water percentage indicated at the end of the slurry preparation step, formed by mixing phases A and B, and optionally phase C.

The removal of the solvent is controlled so as to obtain the final percentage of water indicated in the tables below.

In the following examples, iron oxides (INCI: IRON OXIDE) treated with a hydrophilic compound according to the preceding description, of different colors are used to obtain the desired shade. More particularly, treated iron oxides of black, yellow or red color are used. It is thus possible to prepare mixtures comprising two or three colors so as to obtain the desired shade. The nature and proportion of each pigment treated in the mixture has no impact on the performance of the product, nor on the properties when it is applied to the skin.

In the examples below, the indicated percentage of treated pigment(s) represents the total amount of treated pigment(s) used, obtained for example by adding the percentages of each color used in the mixture.

### Example 1: Selection of Pigments Treated with an Additive of Formula (I): Sodium Glycerophosphate (Otherwise Called ‘SGP Pigments’) and Phytic Acid (Otherwise Called ‘PHY Pigments’)

The Applicant has tested several hydrophilic treated pigments and shown that SGP pigments, PHY pigments, in particular iron oxides treated with sodium glycerophosphates or phytic acid, such as those marketed by SENSIENT under the name Unipure SGP or PHY, allowed to address the problem of the inhomogeneity of the texture of make-up powders produced by a water slurry process.

TABLE 1

| | | | Control B | Invention | Invention |
|---|---|---|---|---|---|
| | | Control A | (silica | (SGP | (PHY |
| | | (untreated | treated | treated | treated |
| | Ingredients | pigments) | pigments) | pigments) | pigments) |
| A | Phenoxyethanol | 0.9 | | | |
| A | Pentylene Glycol | 2.5 | | | |
| B | Fillers | 55 | | | |
| B | Magnesium Myristate | 2 | | | |
| B | Sorbic acid | Qs | | | |
| | MINERAL PIGMENTS | | | | |
| B | Untreated pigment(s) | 25 | | | |
| | IRON OXIDE (SUNPURO ®) | | | | |
| B | Silica treated pigment(s) | | 25 | | |
| | IRON OXIDE (AND) SILICA | | | | |
| | (SYMPHOLIGHT ®) | | | | |
| B | SGP treated pigment(s) | | 25 | | |
| | IRON OXIDE (AND) SODIUM | | | | |
| | GLYCEROPHOSPHATE (UNIPURE ® | | | | |
| | SGP) | | | | |
| B | PHY Treated Pigments | | 25 | | |
| | IRON OXIDE (AND) PHYTIC ACID | | | | |
| | (AND) SODIUM HYDROXIDE | | | | |
| | (UNIPURE ® PHY) | | | | |
| C | Octyldodecyl Stearoyl Stearate | 14 | | | |
| | (CERAPHYL ® 847) | | | | |
| | EVALUATIONS | | | | |
| | Aspect | − | + | +++ | +++ |
| | Application on the skin | − | − | +++ | +++ |

Phase A: aqueous phase
Phase B: particulate phase (powders)
Phase C: oily phase

### Operating Mode

The ingredients of phase A are dissolved in an excess amount of water such that phase A is approximately equivalent in weight to the particulate solid phase B.

The ingredients of phase B are mixed then dispersed in phase A using a Rayneri; at 1000 revolutions/minutes for 5 to 10 minutes until a homogeneous mixture is obtained.

The binding phase C is added last to the pasty dispersion formed by phases A and B. The slurry is homogenized at 1000 revolutions/minute for 15 minutes.

The top Fill with the silicone molds is filled, taking care to remove any air bubbles.

A fabric covering the composition is placed on the surface of the mold. A pressure of 1 bar is applied for three seconds to the powder slurry, while sucking up the water by creating a vacuum in the enclosure.

The molds are then placed in the freezer for 30 minutes to allow easy unmolding.

After unmolding, the products are placed in an oven (50° C.) for 5 hours or alternatively at 45° C. overnight.

The water is completely removed from the slurry.

### Evaluation

The appearance of the final composition and its application to the skin are evaluated in the laboratory.

Appearance of the composition (cohesion of the compact, absence of marbling):
+++: very satisfactory
+: moderately satisfactory
−: unsatisfactory (lack of cohesion, sandy appearance)

Coverage and comfort when applied:
+++: very satisfactory
+: moderately satisfactory
−: unsatisfactory (lack of coverage and/or roughness when applied)

The results of the comparative evaluation of the control compositions (untreated or silica-treated pigments) and invention (SGP or PHY treated pigments) show that the use of a treatment with an additive of formula (I) as described according to invention allows both to obtain properties of cohesion of the compact and absence of marbling, and properties of coverage and comfort when applied to the skin.

### Example 2: Matte Compact Foundation

The formula of the solid composition according to the invention is presented in the table below (% by weight of the final composition).

TABLE 2

| | | % by |
|---|---|---|
| Phase | Ingredients | weight |
| A | WATER | 43 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.5 |
| A | CAPRYLYL GLYCOL | 0.2 |
| B | MICA | 26 |
| B | BORON NITRIDE | 17.1 |
| B | LAUROYL LYSIN | 6.5 |
| B | CELLULOSE (CELLULOBEAD ®) | 2 |
| B | IRON OXIDE (AND) SODIUM GLYCERO- | 1.9 |
| | PHOSPHATE (UNIPURE ® SGP) | |
| B | TITANIUM DIOXIDE (AND) SILICA | 2.6 |
| | (SPHERICA HC WHITE 30) | |

Phase A: aqueous phase
Phase B: particulate phase (powders)

To prepare the composition according to the table above, the ingredients of Phase A comprising an excess of water are mixed together, which are cold dispersed with a Rayneri at 500 revolutions/minute for 5-10 minutes.

The mixture of powders from phase B is then added to phase A, then the whole is dispersed with a Rayneri, gradually increasing the speed to a maximum of 1200 revolutions/minute to obtain a visually homogeneous mixture.

At this stage, the water is present in excess in the slurry, in an amount of 132% by weight relative to the sum of all the other ingredients of the composition of the invention.

The shaping is carried out by filling, in Top Fill mode, silicone molds equipped with a support grid. A fabric covering the composition is placed on the surface of the mold, then compacted and the solvent of the powder slurry is simultaneously sucked off at 4 bar for 30 seconds.

The mold is placed in the freezer for 1 hour.

After unmolding, the composition is placed in an oven at 45° C. for two hours.

After this drying step, the composition is placed in a sealed pack.

The application of the composition produces a matte and covering effect associated with a freshness effect related to the presence of water in the formula.

### Example 3 Moisturizing Foundation

The formula of the foundation according to the invention is presented in Table 3 below (% by weight of the final composition).

TABLE 3

| | | % by |
|---|---|---|
| Phase | Ingredients | weight |
| A | WATER | 33.3 |
| | GLYCEROL | 10.3 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.6 |
| A | CAPRYLYL GLYCOL | 0.3 |
| B | MICA (MEARLMICA TREAT SVA) | 32.2 |
| B | LAUROYL LYSIN | 6.7 |
| B | CELLULOSE (CELLULOBEAD ®) | 2.3 |
| B | IRON OXIDE (AND) SODIUM GLYCERO- | 2.3 |
| | PHOSPHATE (UNIPURE ® SGP) | |
| B | TITANIUM DIOXIDE (AND) SODIUM | 3 |
| | GLYCEROPHOSPHATE (UNIPURE ® SGP) | |
| B | PEARL (FLAMENCO ULTRA SILK 2500) | 5.5 |
| C | UNDECANE and TRIDECANE (CETIOL ® | 3.3 |
| | ULTIMATE MB) | |

Phase A: aqueous phase
Phase B: particulate phase (powders)
Phase C: oily phase

To prepare the compact foundation f presented in the Table above, the ingredients of Phase A comprising an excess of water are mixed together, which are cold dispersed with a Rayneri at 500 revolutions/minute for 5-10 minutes, are mixed together.

The mixture of powders from the phase is then added to phase A, then the whole is dispersed with a Rayneri, gradually increasing the speed to a maximum of 1200 revolutions/minute to obtain a visually homogeneous mixture.

Phase C is added last to the paste formed by phases A and B. Homogenization is continued until a homogeneous paste is obtained.

At this stage, the water is present in excess in the slurry, in an amount of 107% by weight compared to the sum of all the others of the formula of the composition.

The shaping is carried out by filling, in Top Fill mode, silicone molds equipped with a support grid. A fabric covering the composition is placed on the surface of the mold, then the solvent of the powder slurry is sucked off at 4 bar for 30 seconds.

The mold is placed in the freezer for 1 hour.

After unmolding, the composition is placed in an oven at 45° C. for two hours.

After this drying step, the composition is placed in a sealed pack.

The application of the composition produces a covering effect associated with a moisturizing effect and a fresh effect related to the presence of water in the formula.

### Example 4 Pearly Pink Highlighter

The formula of the solid composition according to the invention is presented in Table 4 below (% by weight of the final composition):

TABLE 4

| | | % by |
|---|---|---|
| Phase | Ingredients | weight |
| A | WATER | 31 |
| | GLYCEROL | 10.7 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.7 |
| A | CAPRYLYL GLYCOL | 0.3 |
| B | PEARLS | 39.1 |
| B | LAUROYL LYSIN | 1.4 |
| B | CELLULOSE (CELLULOBEAD ®) | 2.4 |
| B | IRON OXIDE (AND) SODIUM GLYCERO- | 0.2 |
| | PHOSPHATE (UNIPURE ® SGP) | |
| B | TITANIUM DIOXIDE (AND) SILICA | 7.3 |
| | (SPHERICA HC WHITE 30) | |
| B | CALCIUM ALUMINUM BOROSILICATE | 4.6 |
| C | UNDECANE and TRIDECANE (CETIOL ® | 2.1 |
| | ULTIMATE MB) | |

Phase A: aqueous phase
Phase B: particulate phase (powders)
Phase C: oily phase

To prepare the highlighter presented in the Table above, the ingredients of Phase A comprising an excess of water, which are cold dispersed with a Rayneri at 500 revolutions/minute for 5-10 minutes, are mixed together.

The mixture of powders from phase B is then added to phase A, then the whole is dispersed with a Rayneri, gradually increasing the speed to a maximum of 1200 revolutions/minute.

Phase C is added last to the paste formed by phases A and B. Homogenization is continued until a homogeneous paste is obtained.

At this stage, the water is present in excess in the slurry, in an amount of 74% by weight compared to the sum of all the other ingredients of the mixture.

The shaping is carried out by filling, in Top Fill mode, silicone molds equipped with a support grid. A fabric covering the composition is placed on the surface of the mold, then a step of compression (Pressure=2 bar) and suction of the solvent (Pressure=2 bar) is carried out for 20 seconds.

The mold is placed in the freezer for 1 hour.

After unmolding, the composition is placed in an oven at 45° C. for one hour.

After the drying step, the composition is placed in a sealed pack.

### Hardness Measurement

The hardness is measured according to the following protocol:

The hardness is measured using a TA.XT PLUS TEXTURE ANALYSER (STABLE MICROSYSTEMS) texturometer, equipped with a rod on which is fixed a cylinder with a diameter of 5 mm which is applied to the surface of the compact. The cylinder penetration force is measured over a thickness of 1 mm at a speed of 0.5 mm/second.

The average hardness of the composition of this example 4, measured on two parts, according to the protocol previously described, is equal to 316 grams force (Gf).

The pearly pink highlighter has a moisturizing effect on the skin.

### Example 5 Eyeshadow

A brown eye shadow is prepared.

The formula of the composition is presented in Table 5 below (% by weight of the final composition):

TABLE 5

| | | % by |
|---|---|---|
| Phase | Ingredients | weight |
| A | PENTYLENE GLYCOL | 2.5 |
| A | PHENOXYETHANOL | 0.9 |
| B | TALC | 54.2 |
| B | IRON OXIDE (AND) SODIUM GLYCERO- | 25.1 |
| | PHOSPHATE (UNIPURE ® SGP) | |
| B | MAGNESIUM MYRISTATE | 2 |
| B | SORBIC ACID | 0.3 |
| C | ISONONYL ISONONANOATE | 8 |
| C | ISONONYL ISONONANOATE (AND) ETHYLENE | 7 |
| | /PROPYLENE/STYRENE | |
| | COPOLYMER (AND) BUTYLENE/ETHYLENE/ | |
| | STYRENE COPOLYMER (AND) | |
| | BHT (VERSAGEL ® MN 1600) | |

Phase A: aqueous phase
Phase B: particulate phase (powders)
Phase C: oily phase

To prepare the eyeshadow presented in the Table above, the ingredients of Phase A comprising an excess of water are mixed.

The mixture of powders from phase B is then added to phase A, then the whole is cold dispersed with a Rayneri at 1000 revolutions/minute for 15 minutes.

Phase C is added, then dispersed again with a Rayneri at 1000 revolutions/minute for 15 minutes until a homogeneous slurry is obtained.

At this stage, the water is present in the slurry, in an amount of 50% by weight relative to the sum of all the other ingredients in the mixture.

The shaping is carried out by filling, in Top Fill mode, silicone molds equipped with a support grid.

A fabric covering the formula is placed on the surface of the mold then a solvent suction and compacting step (pressure=1 bar) is carried out for 3 seconds. The mold is placed in the freezer for 30 minutes.

After demolding, the composition is placed in an oven at 45° C. for one night.

After complete drying (the final composition is anhydrous), the composition is placed in a suitable pack.

The application of eyeshadow produces a covering effect.

### Example 6 Pearl Bronzer

A solid compact powder having a pearlescent effect on the skin is prepared.

The formula of the composition is presented in Table 6 below (% by weight of the final composition):

TABLE 6

| | | % by |
|---|---|---|
| Phase | Ingredients | weight |
| A | WATER | 31 |
| | GLYCEROL | 10.7 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.6 |
| A | CAPRYLYL GLYCOL | 0.3 |
| B | PEARLS | 40.3 |
| B | LAUROYL LYSIN | 1.7 |
| B | CELLULOSE (CELLULOBEAD ®) | 2.4 |
| B | IRON OXIDE (AND) PHYTIC ACID | 4.4 |
| | (AND) SODIUM HYDROXIDE | |
| | (UNIPURE ® PHY) | |
| B | TITANIUM DIOXIDE (AND) SILICA | 6.3 |
| | (SPHERICA HC WHITE 30) | |
| C | UNDECANE and TRIDECANE (CETIOL ® | 2.1 |
| | ULTIMATE MB) | |

Phase A: aqueous phase
Phase B: particulate phase (powders)
Phase C: oily phase

To prepare the pearly bronzer shown in the table above, the ingredients of Phase A comprising an excess of water are mixed together and cold dispersed with a Rayneri at 500 revolutions/minutes for 5-10 minutes.

The mixture of powders of the phase is then added to phase A, then the whole is dispersed with a Rayneri, gradually increasing the speed to a maximum of 1200 revolutions/minute.

Phase C is added last, continuing the homogenization.

At this stage, the water is present in excess in the slurry, in an amount of 83% by weight compared to the sum of all the other ingredients of the mixture.

The shaping is carried out by filling, in Top Fill mode, silicone molds equipped with a support grid. A fabric covering the formula is placed on the surface of the mold, then a step of compression (Pressure=2 bar) and suction of the solvent (Pressure=1 bar) is carried out for 20 seconds.

The mold is placed in the freezer for 1 hour.

After unmolding, the composition is placed in a sealed pack.

The application of the composition produces a pearly effect coupled with a freshness effect related to the presence of water in the formula.

### Example 7 Eye Shadow for Care

An eyeshadow suitable for skin care is prepared.

The formula of the composition is presented in Table 7 below (% by weight of the final composition):

TABLE 7

| | | % by |
|---|---|---|
| Phase | Ingredients | weight |
| A | WATER | 37.6 |
| A | GLYCEROL | 9.7 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.6 |
| A | CAPRYLYL GLYCOL | 0.2 |
| B | LAUROYL LYSIN | 1.2 |
| B | CELLULOSE (CELLULOBEAD ®) | 2.2 |
| B | IRON OXIDE (AND) SODIUM GLYCERO- | 6.3 |
| | PHOSPHATE (UNIPURE ® | |
| | SGP) | |
| B | MICA (AND) LAUROYL LYSINE (SVA | 21.4 |
| | treated MMEARLMICA ®) | |
| B | PEARLS | 18.7 |
| C | UNDECANE and TRIDECANE (CETIOL ® | 1.9 |
| | ULTIMATE MB) | |

Phase A: aqueous phase
Phase B: particulate phase (powders)
Phase C: oily phase

To prepare the eyeshadow shown in the table above, the ingredients of Phase A comprising an excess of water are mixed together, which are cold dispersed with a Rayneri at 500 revolutions/minute for 5-10 minutes.

The mixture of powders from phase B is then added to phase A, then the whole is dispersed with a Rayneri, gradually increasing the speed to a maximum of 1200 revolutions/minute.

Phase C is added last, continuing the homogenization.

At this stage, the water is present in excess in the slurry, in an amount of 100% by weight compared to the sum of all the other ingredients of the mixture.

The shaping is carried out by filling, in Top Fill mode, silicone molds equipped with a support grid. A fabric covering the composition is placed on the surface of the mold, then a step of compression (Pressure=2 bar) and suction of the solvent (Pressure=1 bar) is carried out for 20 seconds.

The mold is placed in the freezer for 1 hour.

After unmolding, the composition is placed in a sealed pack.

The composition of this example is useful for caring for the skin of the eyelids and provides a cooling effect related to the presence of water in the formula.

### Example 8 Pearly Pink Highlighter

A highlighter having a pearly effect on the skin is prepared.

The formula of the composition is presented in Table 8 below (% by weight of the final composition):

TABLE 8

| | | % by |
|---|---|---|
| Phase | Ingredients | weight |
| A | WATER | 30.4 |
| A | GLYCEROL | 10.8 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.6 |
| A | CAPRYLYL GLYCOL | 0.3 |
| A | PULLULAN | 1.5 |
| B | LAUROYL LYSIN | 1.4 |
| B | CELLULOSE (CELLULOBEAD ®) | 2.4 |
| B | IRON OXIDE (AND) SODIUM GLYCERO- | 0.2 |
| | PHOSPHATE (UNIPURE ® SGP) | |
| B | MICA (AND) LAUROYL LYSINE (SVA | 12.8 |
| | treated MEARLMICA ®) | |
| B | PEARLS | 37.3 |
| C | UNDECANE and TRIDECANE (CETIOL ® | 2.1 |
| | ULTIMATE MB) | |

Phase A: aqueous phase
Phase B: particulate phase (powders)
Phase C: oily phase

To prepare the compact highlighter presented in the Table above, the ingredients of Phase A comprising an excess of water are mixed together, which are cold dispersed with a Rayneri at 500 revolutions/minute for 5-10 minutes.

The powder mixture of phase B is then added to phase A, then the whole is dispersed with a Rayneri, gradually increasing the speed to a maximum of 1200 revolutions/minute.

The oily phase C is added last, continuing the homogenization.

At this stage, the water is present in excess in the slurry, in an amount of 74% by weight compared to the sum of all the other ingredients of the mixture.

The shaping is carried out by filling, in Top Fill mode, silicone molds equipped with a support grid. A fabric covering the composition is placed on the surface of the mold, then a step of compression (Pressure=2 bar) and suction of the solvent (Pressure=1 bar) is carried out for 20 seconds.

The mold is placed in the freezer for 1 hour.

After unmolding, the composition is placed in a sealed pack.

The highlighter of this example is applied to the skin of the face to produce a skin radiance effect coupled with a freshness effect related to the presence of water in the formula. It also has improved staying power related to the presence of pullulan, a hydrophilic polymer that improves the formation of a resistant film on the skin.

### Example 9 Matte Bronzing Powder

A solid compact powder having a matte effect on the skin is prepared.

The formula of the composition is presented in Table 9 below (% by weight of the final composition):

TABLE 9

| | | % by |
|---|---|---|
| Phase | Ingredients | weight |
| A | WATER | 35 |
| A | GLYCEROL | 10 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.6 |
| A | CAPRYLYL GLYCOL | 0.3 |
| C | LAUROYL LYSIN | 6.5 |
| B | CELLULOSE (CELLULOBEAD ®) | 2.3 |
| B | IRON OXIDE (AND) SODIUM GLYCERO- | 4.3 |
| | PHOSPHATE (UNIPURE ® SGP) | |
| B | TITANIUM DIOXIDE (AND) SODIUM | 4.7 |
| | GLYCEROPHOSPHATE (UNIPURE ® SGP) | |
| B | MICA (AND) LAUROYL LYSINE (SVA | 32.9 |
| | treated MEARLMICA ®) | |
| C | UNDECANE and TRIDECANE (CETIOL ® | 3.2 |
| | ULTIMATE MB) | |

Phase A: aqueous phase
Phase B: particulate phase (powders)
Phase C: oily phase

To prepare the solid highlighter presented in the Table above, the ingredients of Phase A comprising an excess of water are mixed together, which are cold dispersed with a Rayneri at 500 revolutions/minute for 5-10 minutes.

The mixture of powders from phase B is then added to phase A, then the whole is dispersed with a Rayneri, gradually increasing the speed to a maximum of 1200 revolutions/minute.

Phase C is added last, continuing the homogenization.

At this stage, the water is present in excess in the slurry, in an amount of 107% by weight compared to the sum of all the other ingredients of the mixture.

The shaping is carried out by filling, in Top Fill mode, silicone molds equipped with a support grid. A fabric covering the composition is placed on the surface of the mold, then a step of compression and suction of the solvent is carried out for 20 seconds.

The mold is placed in the freezer for 1 hour.

After unmolding, the composition is placed in a sealed pack.

The highlighter in the example is applied to the skin of the face and produces a matte effect.

In addition, a skin hydration effect (+11%, 6 hours after removal of the film), which is particularly advantageous for skin care, was measured.

The hydration is measured by corneometry, using a CORNEOMETER CM825 apparatus supplied by the company Courage & Khazaka SN “NUM_SERIE”, which with the person skilled in the art is familiar.

The process for measuring the hydration of the stratum corneum is based on the creation of an electric field on the surface of the skin and the detection, using electrodes, of variations in the dielectric constant induced by the state of hydration of the upper layer of the epidermis (see Measurement of the electrical properties is recognized as an objective method of assessing stratum corneum hydration (E. Berardesca—EEMCO guidance for the assessment of stratum corneum hydration: electrical methods—Skin Research and Technology 1997; 3:126-132).

The control formula or the composition of the invention is applied to the forearm of a volunteer. The amount applied corresponds to 2 μl/cm² of skin. The application area is then protected from friction by placing a metal ring surrounding the application area, fixed to the forearm by adhesive.

A measurement is taken on the application area, before application of the composition, then 6 hours after said application of the composition, leaving a period of 15 minutes for stabilization after removal of the anti-friction protection, before the measurement.

The residual film of the control formula or of the composition of the invention is wiped off from the area of application. The variation in hydration is calculated from the measurements carried out according to the process described above.

### Example 10 Pearly Foundation

A solid foundation having a pearly effect on the skin is prepared.

The formula of the composition is presented in Table 10 below (% by weight of the final composition):

TABLE 10

| | | % by |
|---|---|---|
| Phase | Ingredients | weight |
| A | WATER | 31 |
| | GLYCEROL | 10.7 |
| A | HECTORITE (BENTONE ®) | 0.1 |
| A | MAGNESIUM ALUMINUM SILICATE | 0.1 |
| | (VEEGUM ® HV) | |
| A | PHENOXYETHANOL | 0.6 |
| A | CAPRYLYL GLYCOL | 0.3 |
| B | PEARLS | 40.3 |
| B | LAUROYL LYSIN | 1.7 |
| B | CELLULOSE (CELLULOBEAD ®) | 2.4 |
| B | IRON OXIDE (AND) PHYTIC ACID | 4.4 |
| | (AND) SODIUM HYDROXIDE | |
| | (UNIPURE ® PHY) | |
| B | TITANIUM DIOXIDE (AND) SILICA | 6.3 |
| | (SPHERICA HC WHITE 30) | |
| C | UNDECANE and TRIDECANE (CETIOL ® | 2.1 |
| | ULTIMATE MB) | |

To prepare the foundation presented in the Table above, the ingredients of Phase A comprising an excess of water are mixed together and cold dispersed with a Rayneri at 500 revolutions/minute for 5-10 minutes.

The mixture of powders from phase B is then added to phase A, then the whole is dispersed with a Rayneri, gradually increasing the speed to a maximum of 1200 revolutions/minute.

Phase C is added last while continuing to homogenize the paste.

At this stage, the water is present in excess in the slurry, in an amount of 83% by weight compared to the sum of all the other ingredients of the mixture.

The shaping is carried out by filling, in Top Fill mode, silicone molds equipped with a support grid. A fabric is placed, then a step of compression (Pressure=2 bar) and suction of the solvent (Pressure=1 bar) is carried out for 20 seconds

The mold is placed in the freezer for 1 hour.

After unmolding, the composition is placed in a sealed pack.

The application of the composition to the face produces a pearly effect coupled with a freshness effect related to the presence of water in the formula.

### Example 11 Matte Pink Highlighter

A highlighter having a matte finish on the skin is prepared.

The formula of the composition is presented in Table 11 below (% by weight of the final composition):

TABLE 11

| | | % by |
|---|---|---|
| Phase | Ingredients | weight |
| A | WATER | 32.2 |
| A | GLYCEROL | 10.5 |
| A | HECTORITE (BENTONE ®) | 0.1 |
| A | MAGNESIUM ALUMINUM SILICATE | 0.1 |
| | (VEEGUM ® HV) | |
| A | ETHYLHEXYLGLYCERIN | 0.5 |
| A | HYDROXYACETOPHENONE | 0.1 |
| A | CHLORPHENESIN | 0.3 |
| A | CAPRYLYL GLYCOL | 0.5 |
| A | 2.3 HEXANEDIOL1.0 | 1.0 |
| B | LAUROYL LYSIN | 1.3 |
| B | CELLULOSE (CELLULOBEAD ®) | 2.4 |
| B | IRON OXIDE (AND) SODIUM GLYCERO- | 0.2 |
| | PHOSPHATE (UNIPURE ® SGP) | |
| | PEARLS | 36.2 |
| B | MICA (MICA H) | 12.6 |
| C | UNDECANE and TRIDECANE (CETIOL ® | 2.0 |
| | ULTIMATE MB) | |

Phase A: aqueous phase
Phase B: particulate phase (powders)
Phase C: oily phase

To prepare the compact highlighter presented in Table 11 above, the ingredients of Phase A comprising an excess of water are mixed together, which are cold dispersed with a Rayneri at 500 revolutions/minute for 5-10 minutes.

The mixture of powders from phase B is then added to phase A, then the whole is dispersed with a Rayneri, gradually increasing the speed to a maximum of 1200 revolutions/minute.

Phase C is added last, continuing the homogenization.

At this stage, the water is present in excess in the slurry, in an amount of 88% by weight compared to the sum of all the other ingredients of the mixture.

The shaping is carried out by filling, in Top Fill mode, silicone molds equipped with a support grid. A fabric covering the composition is placed on the surface of the mold, then a step of compression (Pressure=2 bar) and suction of the solvent (Pressure=1 bar) is carried out for 20 seconds.

The mold is placed in the freezer for 1 hour.

After unmolding, the composition is placed in a sealed pack.

The highlighter in the example is applied to the skin of the face for a rosy matte finish coupled with a freshness effect related to the presence of water in the formula. The matt finish is related to the use of Mica H in the powder mixture.

1. A solid cosmetic composition for caring for and/or making up keratin materials, in the form of a compact powder comprising, in a physiologically acceptable medium:
a solid phase comprising at least
fillers and
pigments treated with an additive of formula (I) 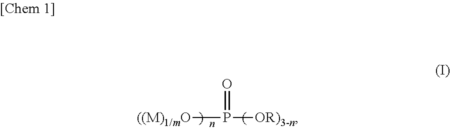
in which:
n represents 1 or 2,
M represents H or a cation,
m represents 1 when M is H and m represents the valence of the cation when M is a cation,
R represents:
a group G selected from a saccharide or a group —[CH2-CHR1-O]q-R2 or —[CH2-CH(CH2OH)—O]q-R2 where:
q represents an integer from 1 to 1000,
for each CH2-CHR1-O unit, R1 independently represents H or a methyl,
R2 represents H or an alkyl comprising from 1 to 3 carbon atoms, and,
a hydrocarbon chain comprising from 1 to 500 carbon atoms substituted by one or more G, phosphate (of formula OPO3(M)_{2/m}) and/or hydroxyl (OH) groups,
said composition being capable of being obtained by a process comprising the following steps: (i) mixing a liquid phase comprising at least water and the solid phase in powder form, so as to form a paste; and (ii) shaping the paste by compacting and total or partial removal of the liquid phase so as to obtain the composition in the form of a compact powder.

2. The cosmetic composition according to claim 1, wherein the pigments are treated with an additive of formula (II) 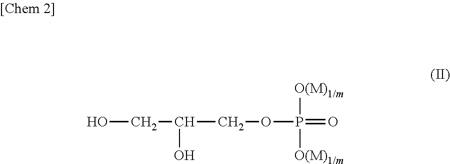
in which
M and m are as defined in claim 1,
n represents 2, R represents a group G of formula-[CH2-CH(CH2OH)—O]q-R2 where q represents 1 and R2 represents H,
and preferably an additive of formula (III) named sodium glycerophosphate (SGP)

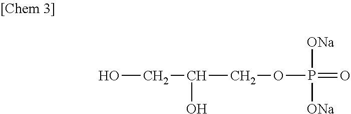

3. The cosmetic composition according to claim 1, wherein the pigments are treated with an additive of formula (V) 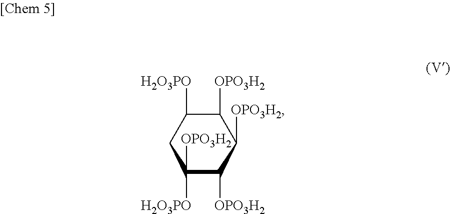
in which
M and m are as defined in claim 1,
n represents 2, R represents a cyclohexyl hydrocarbon chain substituted in positions 2, 3, 4, 5 and 6 by a phosphate group of formula OPO3H2
preferably the additive is phytic acid of formula (V′)

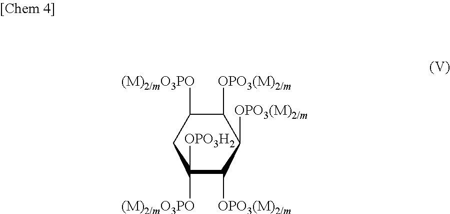

4. The cosmetic composition according to claim 1, wherein said pigments treated with an additive (I), (II), (III), (V) or (V′) are present in the composition in a content ranging from 0.5% to 60% by weight relative to the total weight of said composition.

5. The cosmetic composition according to claim 1, wherein the fillers are selected from platelet fillers, spherical fillers and mixtures thereof.

6. The cosmetic composition according to claim 1, wherein it additionally comprises suspending agents, and optionally cohesion agents.

7. The cosmetic composition according to claim 1, characterized in that wherein it is in the form of a care powder or a make-up powder for the face or the lips.

8. The cosmetic composition according to claim 1, wherein it has 3D relief patterns on the surface.

9. A process for the manufacture of a cosmetic composition as defined in claim 1, wherein it comprises the following steps: (i) mixing a liquid phase comprising at least water and the solid phase in powder form comprising the fillers and and the treated pigments, so as to form a paste; and (ii) shaping the paste, by compacting and total or partial removal of the liquid phase present in the paste.

10. A cosmetic process for caring for and/or making up keratin materials, comprising the application to the keratin materials of a composition as defined according to claim 1.

11. The cosmetic process according to claim 10, wherein the composition is applied onto the skin.

12. The cosmetic composition according to claim 4, wherein said pigments are treated with a sodium glycerophosphate (III) or phytic acid (V).

13. The cosmetic composition according to claim 1, wherein said pigments are iron oxides.

14. The cosmetic composition according to claim 7, wherein the composition is a blush, an eye shadow, a highlighter, a bronzer, a protective foundation powder, a concealer, a lip powder, or an eyebrow powder.

15. The cosmetic composition according to claim 7, wherein the composition is a foundation powder, a highlighter, a blush, or an eye shadow.
